(19) United States
(12) Patent Application Publication
(10) Pub. No.: US 2013/0245632 A1
(43) Pub. Date: Sep. 19, 2013

(54) METHOD AND APPARATUS FOR INSERTION OF AN ELONGATE PIN INTO A SURFACE

(71) Applicants: Joseph P. Iannotti, Strongsville, OH (US); Wael K. Barsoum, Bay Village, OH (US); Jason A. Bryan, Avon Lake, OH (US)

(72) Inventors: Joseph P. Iannotti, Strongsville, OH (US); Wael K. Barsoum, Bay Village, OH (US); Jason A. Bryan, Avon Lake, OH (US)

(73) Assignee: THE CLEVELAND CLINIC FOUNDATION, Cleveland, OH (US)

(21) Appl. No.: 13/613,511

(22) Filed: Sep. 13, 2012

Related U.S. Application Data
(60) Provisional application No. 61/534,152, filed on Sep. 13, 2011.

(51) Int. Cl. A61B 17/17

(52) U.S. Cl. CPC A61B 17/17
USPC 606/96

(57) ABSTRACT

An apparatus for dictating an insertion trajectory and location for insertion of an elongate pin into a surface includes a primary leg having longitudinally spaced proximal and distal primary leg ends. At least one subordinate leg has longitudinally spaced proximal and distal subordinate leg ends. The proximal subordinate leg end is movably attached to the primary leg by a coupling mechanism allowing at least two degrees of freedom of motion of the subordinate leg relative to the primary leg. The distal subordinate leg end is configured for contact with the surface concurrently with contact of the distal primary leg end with the surface, to support the apparatus during use of the apparatus. The primary leg guides longitudinal translational movement of the elongate pin to insertion into the surface at the dictated insertion trajectory and location when the apparatus is supported in a predetermined guiding relationship with the surface.

### RELATED APPLICATION

This application claims priority from U.S. Provisional Application No. 61/534,152, filed 13 Sep. 2011, the subject matter of which is incorporated herein by reference in its entirety.

### TECHNICAL FIELD

The present invention relates to an apparatus and method for use of an insertion tool and, more particularly, to an apparatus for dictating trajectory and location for insertion of an elongate pin into a surface.

### BACKGROUND OF THE INVENTION

In the installation of a prosthetic hip joint into a patient's body, an acetabular component is implanted into the acetabulum of the patient's pelvis. An obverse surface of the acetabular component is configured for articulating contact with a femoral component carried by the patient's femur. A reverse surface of the acetabular component is secured to the bone surface of the acetabulum.

Because the hip prosthesis is normally provided to correct a congenital or acquired defect of the native hip joint, the acetabulum often exhibits a pathologic, nonstandard anatomic configuration. A surgeon must compensate for such pathologic acetabular anatomy when implanting the acetabular component in striving to achieve a solid anchoring of the acetabular component into the acetabulum. Detailed preoperative planning, using two- or three-dimensional internal images of the hip joint, often assists the surgeon in compensating for the patient's anatomical limitations. During the surgery, an elongated pin may be inserted into the surface of the patient's bone, at a trajectory and location that are either predetermined or arbitrarily chosen, to act as a passive landmark or active guiding structure in carrying out the preoperatively planned implantation. This “guide pin” may remain as a portion of the implanted prosthetic joint or may be removed before the surgery is concluded. This type of pin-guided installation is common in any joint replacement procedure—indeed, in any type of surgical procedure in which a surgeon-placed fixed landmark is desirable.

In addition, and again in any type of surgical procedure, modern minimally invasive surgical techniques may dictate that only a small portion of the bone or other tissue surface being operated upon is visible to the surgeon. Depending upon the patient's particular anatomy, the surgeon may not be able to precisely determine the location of the exposed area relative to the remaining, obscured portions of the bone through mere visual observation. Again, a guide pin may be temporarily or permanently placed into the exposed bone surface to help orient the surgeon and thereby enhance the accuracy and efficiency of the surgical procedure.

A carefully placed guide pin, regardless of the reason provided, will reduce the need for intraoperative imaging in most surgical procedures and should result in decreased operative time and increased positional accuracy, all of which are desirable in striving toward a positive patient outcome.

### SUMMARY OF THE INVENTION

In an embodiment of the present invention, an apparatus for dictating an insertion trajectory and an insertion location for insertion of an elongate pin into a surface is described. A primary leg has longitudinally spaced proximal and distal primary leg ends. The distal primary leg end is configured for direct contact with the surface. At least one subordinate leg has longitudinally spaced proximal and distal subordinate leg ends. The proximal subordinate leg end is movably attached to the primary leg by a coupling mechanism allowing at least two degrees of freedom of motion of the subordinate leg relative to the primary leg. The distal subordinate leg end is configured for contact with the surface concurrently with contact of the distal primary leg end with the surface, to support the apparatus in a predetermined guiding relationship with the surface during use of the apparatus. The subordinate leg is adjusted relative to the primary leg to impart the dictated insertion trajectory and insertion location to the apparatus. The primary leg guides longitudinal translational movement of the elongate pin to insertion into the surface at the dictated insertion trajectory and insertion location when the apparatus is being supported in the predetermined guiding relationship with the surface.

In an embodiment of the present invention, a method for inserting an elongate pin into a bone surface is described. A primary leg having longitudinally spaced proximal and distal primary leg ends is provided. At least one subordinate leg is provided. Each subordinate leg has longitudinally spaced proximal and distal subordinate leg ends. The proximal subordinate leg end is movably attached to the primary leg by a coupling mechanism allowing at least two degrees of freedom of motion of the subordinate leg relative to the primary leg. The subordinate leg is adjusted in at least two degrees of freedom relative to the primary leg to impart the dictated insertion trajectory and insertion location to the primary leg. The bone surface is contacted with the distal primary leg end. The bone surface is contacted with the distal subordinate leg end concurrently with contact of the distal primary leg end with the bone surface. The apparatus is supported in a predetermined guiding relationship with the bone surface during use of the apparatus via contact between the distal primary and subordinate leg ends with the bone surface. At least one of an insertion location and an insertion trajectory of the elongate pin relative to the bone surface is dictated by maintaining the primary leg in a predetermined position relative to the bone surface. With the primary leg, longitudinal translational movement of the elongate pin to insertion into the surface is guided at the dictated insertion trajectory and insertion location when the apparatus is being supported in the predetermined guiding relationship with the surface. The bone surface is contacted with a distal end of the elongate pin at the insertion location. The distal end of the elongate pin is inserted into the bone surface along the insertion trajectory.

### BRIEF DESCRIPTION OF THE DRAWINGS

For a better understanding of the invention, reference may be made to the accompanying drawings, in which:

FIG. 1 is a side view of one embodiment of the present invention;

FIG. 2 is a front view of the embodiment of FIG. 1;

FIG. 3 is a rear view of the embodiment of FIG. 1;

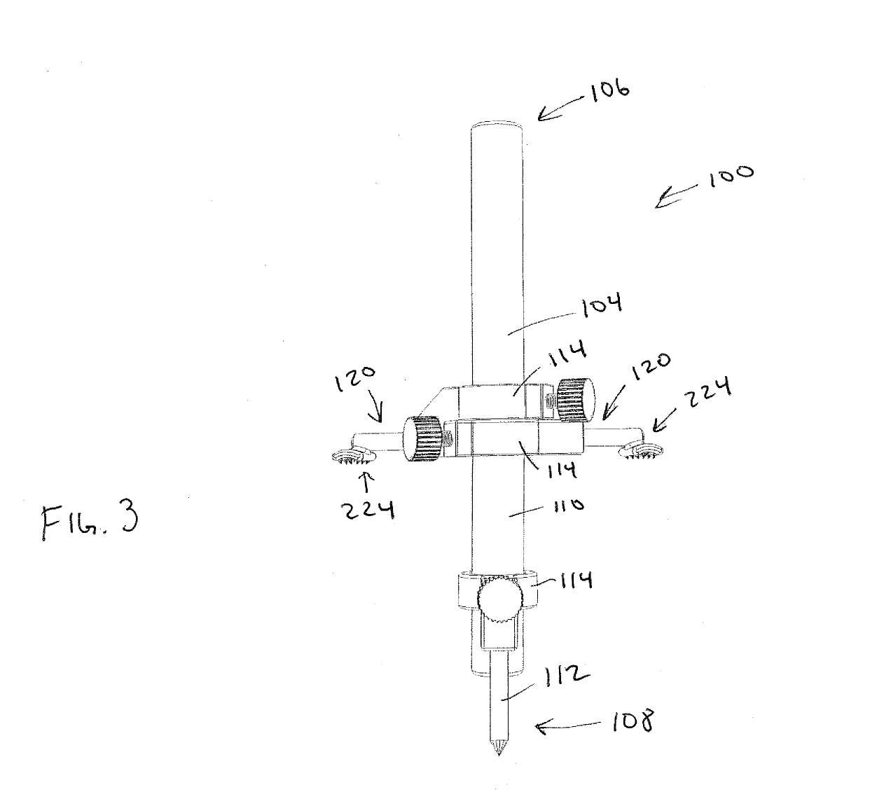

FIG. 4 is a top view of the embodiment of FIG. 1;

FIG. 5 is a bottom view of the embodiment of FIG. 1;

FIG. 6 is a perspective bottom view of the embodiment of FIG. 1;

FIG. 7 is a side view of the embodiment of FIG. 1 having an optional configuration;

FIG. 8 is a side view of the embodiment of FIG. 1 having other optional configurations; and

FIG. 9 is a schematic side view of the embodiment of FIG. 1 in an example use environment.

### DESCRIPTION OF EMBODIMENT

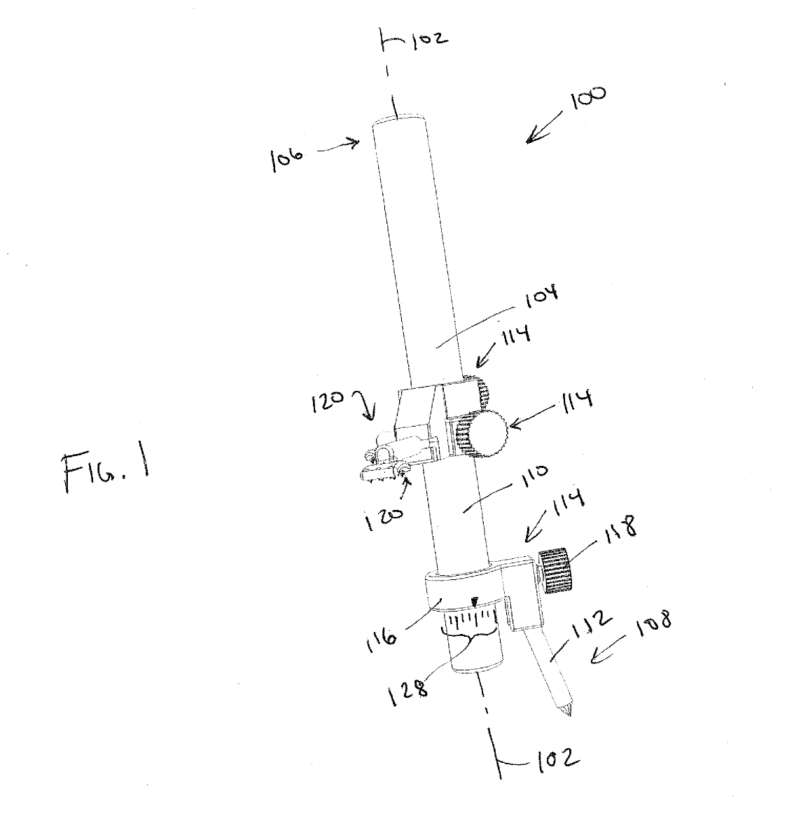

In accordance with the present invention, FIG. 1 depicts an apparatus 100 for dictating an insertion trajectory 102 and an insertion location for insertion of an elongate pin (not shown) into a surface. The surface is shown and described herein at least as a patient tissue (here, an acetabulum), but the patient tissue could be any desired type such as, but not limited to, hip joints, shoulder joints, knee joints, ankle joints, phalangeal joints, metatarsal joints, spinal structures, long bones (e.g., fracture sites), other bones or soft tissues, or any other suitable body tissue of a patient for the present invention. The apparatus 100, or portions thereof, may be sterilizable or otherwise reusable for multiple patients.

A primary leg 104 has longitudinally spaced (i.e., longitudinal with respect to the primary leg) proximal and distal primary leg ends 106 and 108, respectively. The distal primary leg end 108 is configured for direct contact with the surface, such as during insertion of the elongate pin into the surface. In the apparatus 100 shown in the Figures, the primary leg 104 has a principal body 110 and a stabilizing limb 112 branching outward at an angle from the principal body of the primary leg. The elongate pin may be guided to the dictated insertion trajectory and insertion location by the principal body 110 of the primary leg 104, as described below.

In the apparatus 100 depicted in the Figures, the stabilizing limb 112 includes the distal primary leg end 108 (i.e., the portion of the primary leg 104 which contacts the surface) at a location spaced apart from the principal body 110 of the primary leg, though it is also contemplated that the distal primary leg end may instead be a portion of the principal body. The stabilizing limb 112, when present, may be attached to the principal body 110 in a fixed relationship or, as shown in the Figures, may be movably attached to the principal body via a mechanism such as the coupling mechanism 114 shown. This coupling mechanism 114 has a collar 116 which can be slid up and down (i.e., substantially longitudinally with respect to the primary leg 104) on the principal body 110 and/or rotated around the principal body. The coupling mechanism 114 also has a turnscrew 118 that can be tightened to hold the collar 116, and thus the attached stabilizing limb 112, in a predetermined relationship with the principal body 110. In this way, the stabilizing limb 112 can be adjusted in at least two degrees of freedom with respect to the principal body 110. The six degrees of freedom available to an untethered structure are translational movement along each of the X, Y, and Z axes, as well as rotation about each of those axes—i.e., roll movement about the X-axis, pitch movement about the Y-axis, and yaw movement about the Z-axis.

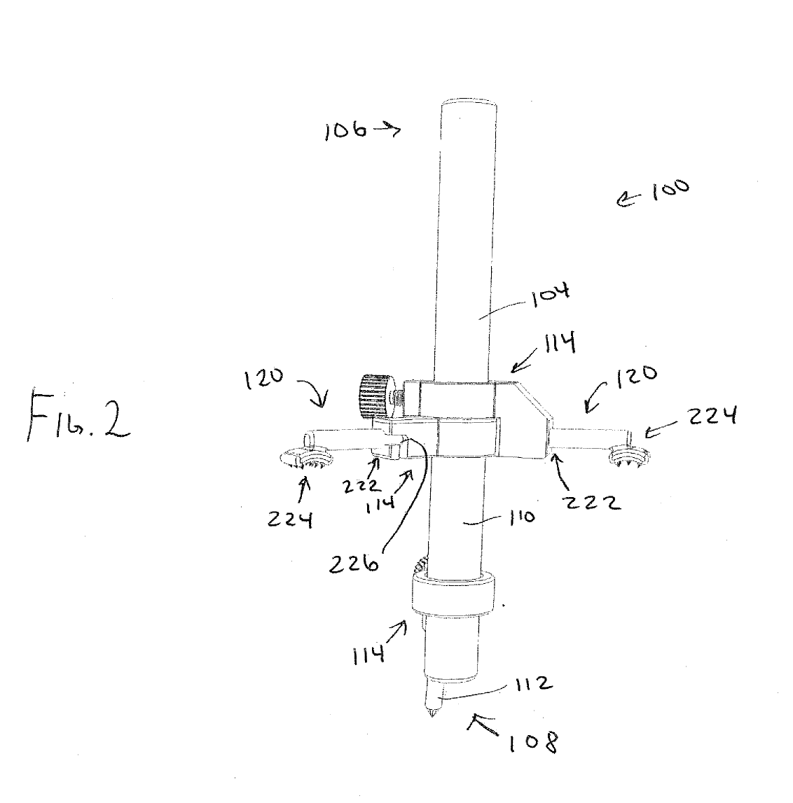

At least one subordinate leg 120 (two shown in the Figures) may be provided to the apparatus 100. As can be seen in FIG. 2, each subordinate leg 120 has longitudinally spaced (i.e., longitudinal with respect to the subordinate leg) proximal and distal subordinate leg ends 222 and 224, respectively. Each proximal subordinate leg end 222 may be movably attached to the primary leg 104 by a coupling mechanism 114 (similar to that used with the stabilizing limb 112) which allows at least two degrees of freedom of motion of the subordinate leg 120 relative to the primary leg. Here, the coupling mechanisms 114 allow translational movement of the subordinate legs 120 along the primary leg 104, as well as allowing rotational movement of the subordinate legs about the primary leg. Optionally, a subordinate leg 120 may be connected to the coupling mechanism 114 for pivoting movement of the subordinate leg with respect to the primary leg 104. This pivoting movement, when present, may be facilitated by a ball-and-socket joint (not shown) or other mechanism allowing the subordinate leg 120 to trace a conical range of motion with an apex of the cone at or near the primary leg 104. Another pivoting movement which may be facilitated by an appropriately-designed coupling mechanism 114 could be rotation of the subordinate leg 120 about its own longitudinal axis.

The subordinate leg(s) 120 may have a variable length. As shown in FIG. 2, a release slot 226 or other appropriate structure may be provided to allow a particular subordinate leg 120 to be readily removed from the coupling mechanism 114 and a substitute subordinate leg (not shown), having a different length or another structural difference from the particular subordinate leg, can then be attached to the coupling mechanism as desired. Additionally or alternatively, the subordinate leg 120 itself may include a telescoping mechanism or other structure to allow the length (or any other dimension) of the subordinate leg to vary as desired.

Optionally, the coupling mechanism 114 may include at least one detent feature (not shown) configured to facilitate discrete manual adjustment of the attached subordinate leg 120 or stabilizing limb 112 relative to the primary leg 104 in at least one degree of freedom. For example, a ratcheting or keyed structure, frictional engagement, or any other suitable means may be provided to restrict rotation and/or translation of the coupling mechanism 114 with respect to the primary leg 104. One of ordinary skill in the art can readily provide a suitable detent feature for a particular application of the present invention. A visual and/or tactile indicator, such as the rotation scale 128 shown in FIG. 1, could provide a discrete indication of a position of the coupling mechanism 114 with respect to the primary leg 104 or another structure of the apparatus 100, in cooperation with or instead of any detent feature provided to the apparatus.

As shown in the Figures, the primary leg 104 may be substantially larger in at least two dimensions (here, in all three dimensions of width, length, and depth) than corresponding dimensions of at least one subordinate leg 120.

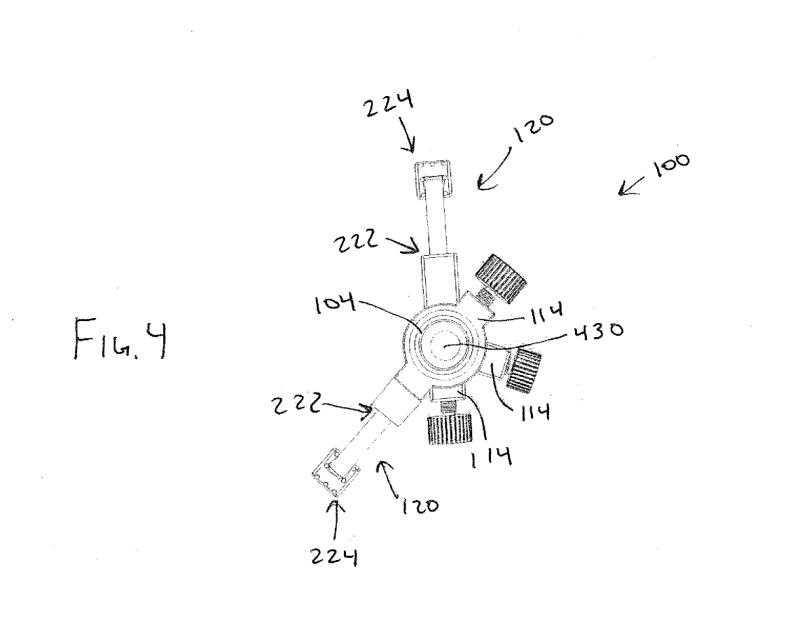

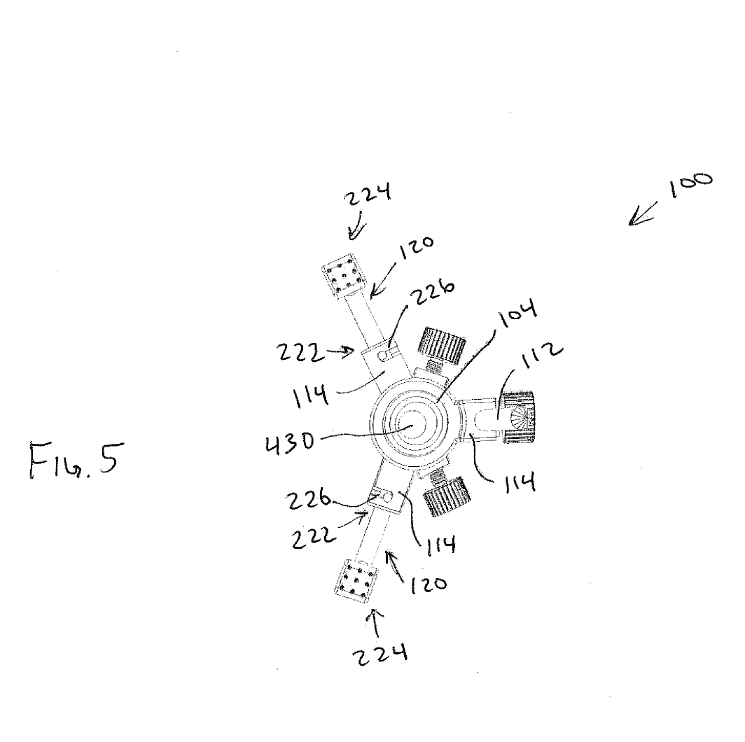

FIGS. 4 and 5 are top and bottom views, respectively, of the apparatus 100. As can be seen here, the primary leg 104 includes a guiding passageway 430 extending longitudinally at least partway therethrough—as depicted in the Figures, the guiding passageway is a bore extending longitudinally entirely through the primary leg and having a tubular configuration with a closed-circle cross-sectional shape. Other suitable guiding passageways 430 include, but are not limited to, a passageway having a non-circular closed cross-sectional shape; a passageway extending longitudinally down a trough or groove in the primary leg 104 (i.e., a bore having an open cross-sectional shape); a series of longitudinally spaced rings, hooks, or other discrete guiding structures (e.g., a fishing rod type guiding configuration); a rail configured to engage at least a portion of the elongate pin; or any other suitable arrangement functioning as described. The elongate pin passes through at least a portion of the guiding passageway 430 and is guided thereby to attain the dictated insertion trajectory and/or insertion location with respect to the surface, as described below.

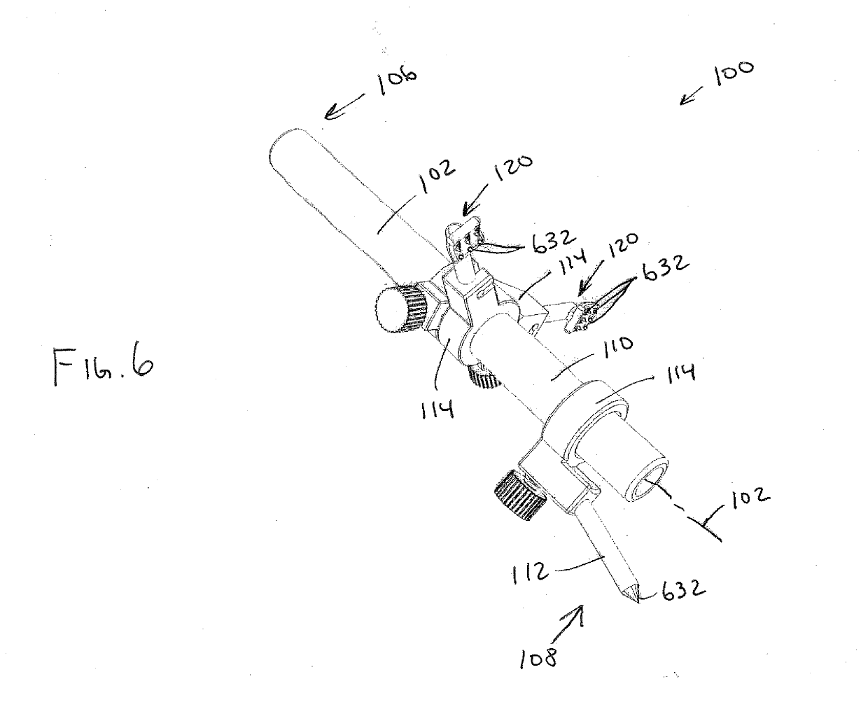

As shown in FIG. 6, at least one of the distal primary leg end 108 and the distal subordinate leg end 224 may include at least one projection 632 which engages with the surface to help maintain position of the respective primary or subordinate leg 104 or 120 with respect to the surface. For example, the projection 632 may penetrate at least a short distance into the surface in a spike-like manner. As another option, the projection 632 may engage the surface--if an engagement beyond mere contact is desirable—in an adhesive, suction, frictional, or any other suitable manner.

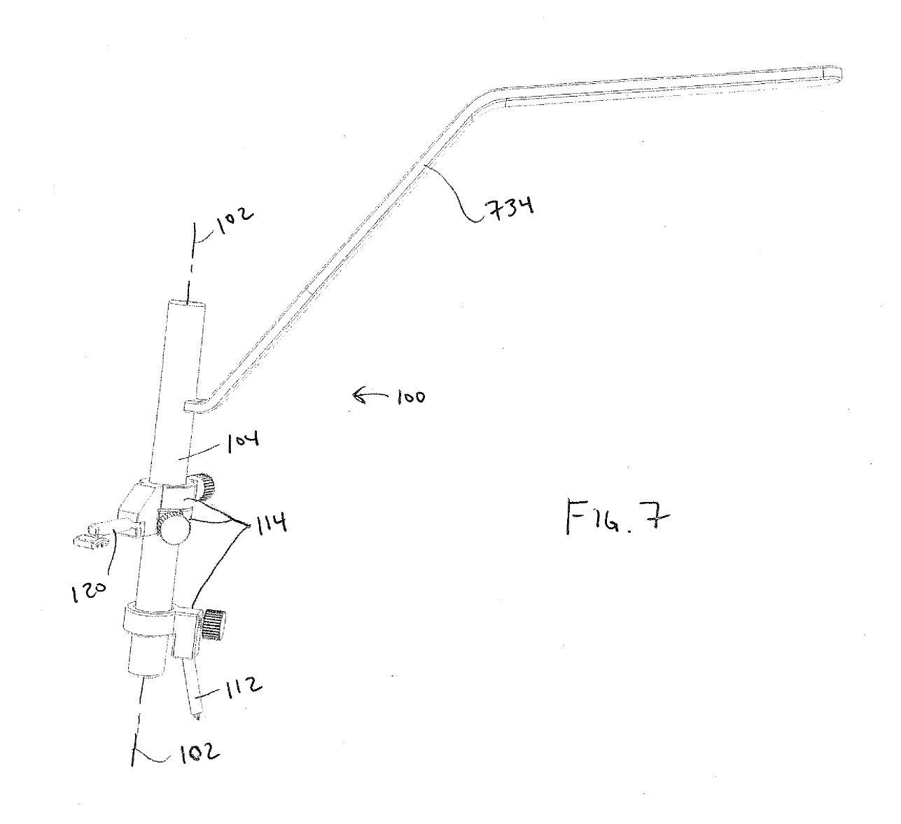

A permanently attached or removable handle 734 may be provided, as shown in FIG. 7, to facilitate manipulation of the apparatus 100. When the apparatus 100 is used in a surgical setting, for instance, the handle 734 may be useful in positioning and/or steadying the apparatus 100 in a desired position while avoiding having yet another person's hand and arm crowding the surgical field. It is contemplated that the handle 734 will usually be attached to the primary leg 104, but one of ordinary skill in the art will be able to configure a handle as desired for a particular application of the present invention and may permanently or removably attach the handle to any desired portion of the apparatus 100.

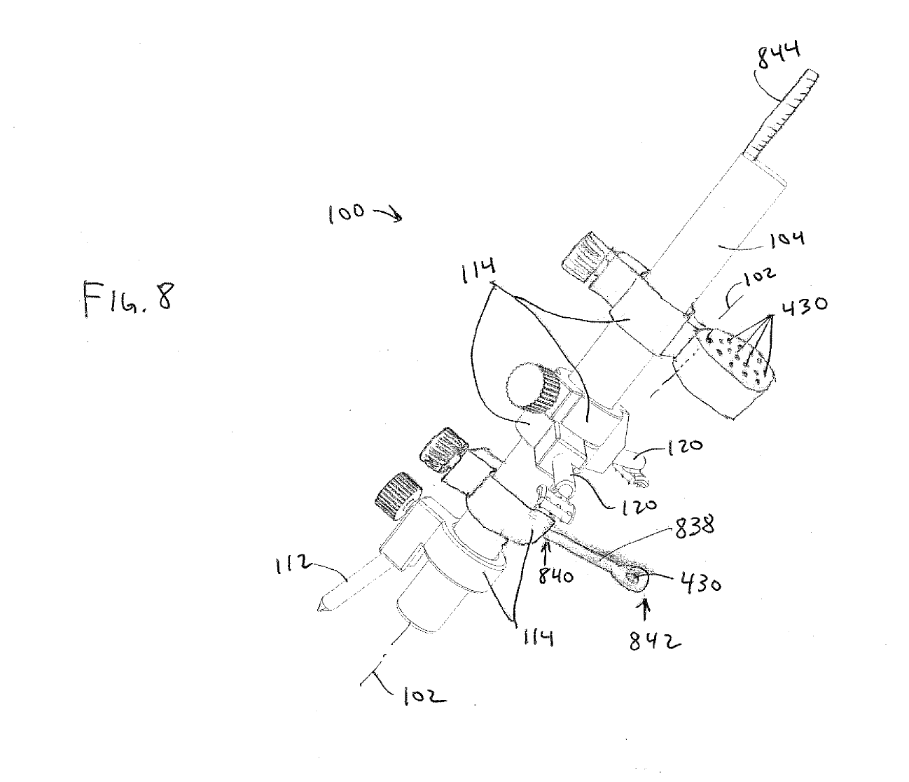

FIG. 8 depicts several options for guiding an elongate pin at the dictated insertion trajectory 102 and/or insertion location. A guiding block 836 is provided to the primary leg 104, optionally through the use of a coupling mechanism 114 as shown. The guiding block 836 includes a plurality of guiding passageways 430 extending longitudinally at least partially therethrough (here, the guiding passageways extend entirely through the guiding block). At least a portion of the elongate pin passes through at least a portion of a chosen one of the plurality of guiding passageways 430 to attain the dictated insertion trajectory (102, only one shown for clarity) and insertion location with respect to the surface. The choice of the chosen one of the plurality of guiding passageways bears a direct positional relationship with the dictated insertion location. The function of the guiding block 836 as a portion of the apparatus 100 may bear similarities to the function of the location block of co-pending U.S. patent application Ser. No. 12/854,362, filed Aug. 11, 2010 and titled “Method and Apparatus for Insertion of an Elongate Pin Into a Surface”, and incorporated herein by reference in its entirety. As another, nondepicted option, the guiding passageways 430 of the guiding block 836 may be integrated into the principal body 110 of the primary leg 104 such that the principal body has a plurality of guiding passageways extending at least partially therethrough and the user can choose one of the plurality of guiding passageways to impart at least one of a desired insertion trajectory 102 and desired insertion location to the elongate pin.

Also depicted in FIG. 8 is an offset guiding extension 838, again provided to the primary leg 104 through the use of a coupling mechanism 114. The offset guiding extension 838 has longitudinally spaced (i.e., longitudinal with respect to the offset guiding extension) proximal and distal extension ends 840 and 842, respectively. The proximal extension end 840 may be movably attached to the primary leg 104 by a coupling mechanism 114 (similar to that used with the stabilizing limb 112 and/or the subordinate leg 120) which allows at least two degrees of freedom of motion of the offset guiding extension 838 relative to the primary leg. A guiding passageway 430 is located at the distal extension end 842 and is configured to guide the elongate pin to the dictated insertion trajectory and insertion location, as described below, instead of or in addition to the guiding of an elongate pin by a guiding passageway 430 formed in or by another portion of the primary leg 104.

The apparatus 100 may include a depth control feature, such as the indication scale 844 shown in FIG. 8. The depth control feature may indicate and/or limit a depth to which the elongate pin is inserted into the surface. For example, the indication scale 844 can be configured to indicate a starting position, when the elongate pin is resting lightly on the surface, and an insertion position, when the elongate pin has been pushed into the surface. The distance between the starting position and the insertion position on the indication scale 844 will reflect the insertion depth of the elongate pin. One of ordinary skill in the art can readily provide another type of depth indicator or depth stop for a particular application of the present invention.

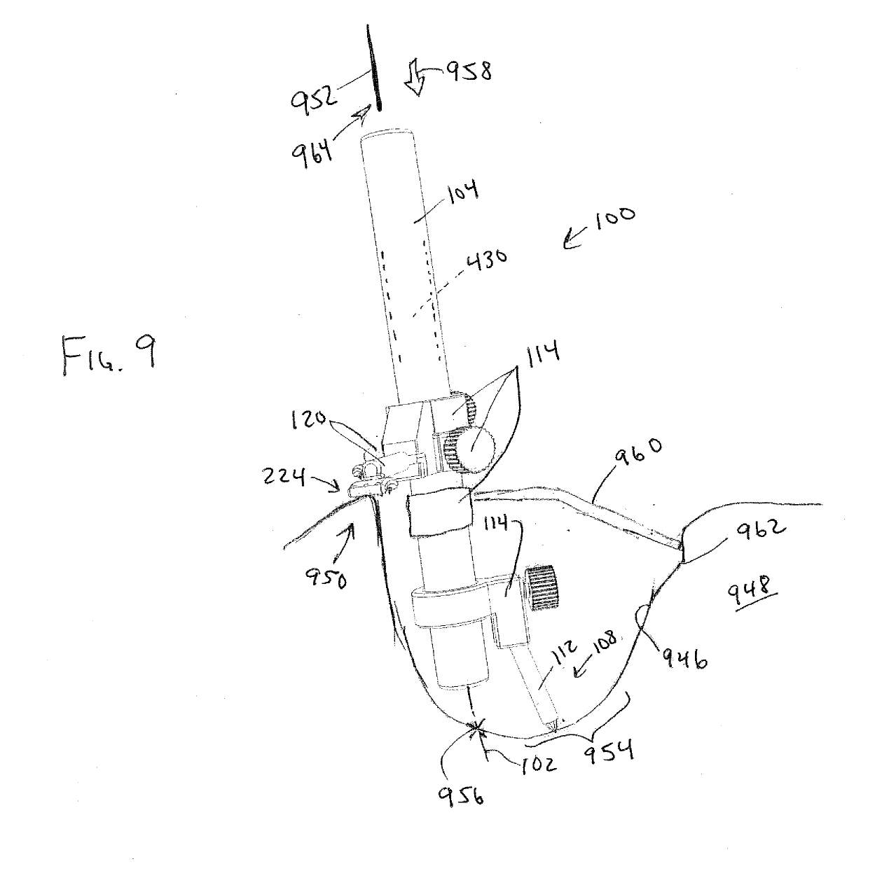

FIG. 9 schematically depicts the apparatus 100 in a first example use environment, the acetabulum 946 of a pelvis 948 of a patient. An acetabular rim 948 surrounds the acetabulum in a known manner. Before the apparatus 100 is placed into the depicted relationship with the acetabulum 946, the distal primary leg end 108 and each of the distal subordinate leg ends 224 (only one visible in the view of FIG. 9) should be adjusted to support the apparatus in a predetermined guiding relationship with the surface (here, the surface of the acetabulum 946) during use of the apparatus. In some embodiments of the apparatus 100, the distal primary leg end 108 will be substantially nonadjustable, and in those embodiments, the distal subordinate leg end(s) 224 will be adjusted to help the apparatus achieve the predetermined guiding relationship with the surface. When there are two subordinate legs 120, those two legs and the primary leg 104 will cooperatively create a tripod support of the apparatus 100 in the predetermined guiding relationship with the surface during use of the apparatus.

The predetermined guiding relationship that the apparatus 100 is configured to achieve with the surface may be predetermined in any suitable manner. It is contemplated, however, that the predetermined guiding relationship will include both contact of the surface with the apparatus 100 and placement of the primary leg 104 to guide an elongate pin (952 in FIG. 9) to insertion into the surface at a dictated insertion trajectory and insertion location when the apparatus is being supported in the predetermined guiding relationship with the surface. To place the primary leg 104 appropriately for this result, each subordinate leg 120 may be adjusted relative to the primary leg 104 to impart the dictated insertion trajectory 102 and insertion location to the apparatus 100.

In the acetabulum 946 example shown in FIG. 9, the distal primary leg end 108 is placed into contact with a fovia 954 of the acetabulum. Each distal subordinate leg end 224 is configured to contact a known point on the pelvis 948 (here, the acetabular rim 950) to support the apparatus in the predetermined guiding relationship with the surface of the acetabulum. Since the subordinate leg(s) 120 are adjustable, however, the user may manually adjust the subordinate leg(s) 120 relative to the primary leg 104 to dictate at least one of the insertion trajectory 102 and the insertion location 956 for the elongate pin 952. For example, the longitudinal distance between the proximal and distal subordinate leg ends 222 and 224--that is, the length of the subordinate leg 120--may be adjustable to help dictate at least one of the insertion trajectory 102 and the insertion location 956 for the elongate pin 952. As another example, substitute subordinate legs (not shown), having different lengths, sizes, shapes, or any other physical property can be provided to the apparatus 100 in lieu of one or more of the original subordinate legs 120 to help dictate at least one of the insertion trajectory 102 and the insertion location 956 for the elongate pin 952. As a further example, the respective coupling mechanisms 114 could be loosened to allow translation and/or rotation of the coupling mechanisms with respect to the primary leg 104 to reposition the subordinate legs 120 as desired. One of ordinary skill in the art could provide other ways for the subordinate legs 120 to be adjusted. The manner in which the primary and/or subordinate legs 104 and 120 are adjusted is not essential to the functioning of the apparatus 100.

Because the insertion trajectory 102 and insertion location 956 generally carry some significance for later use of the elongate pin 952 as a surgical landmark, it is contemplated that the user will generally plan the insertion trajectory and insertion location in advance of the surgery and transfer that pre-planned information to the apparatus 100 in any suitable manner. One way that the insertion trajectory 102 and/or the insertion location 956 may be predetermined by the user is through the use of an external dictator, such as, but not limited to, a preoperative planning system such as that disclosed in co-pending U.S. Provisional Patent Application No. 61/408,392, filed Oct. 29, 2010 and titled “System of Preoperative Planning and Provision of Patient-Specific Surgical Aids”, incorporated herein by reference in its entirety. A preoperative planning system may provide the user with predetermined settings for the adjustable portions of the apparatus 100. In this instance, scales, detents, or other markings or structures may be provided to the apparatus 100 as adjustment aid tools to assist the user with placing the movable structures of the apparatus into the appropriate predetermined positions. As an example, the rotation scale 128 previously discussed could carry a variety of numerical values, and a marking on the coupling mechanism 114 could be lined up with a particular value dictated by the preoperative planning system.

Another way that a predetermined desired elongate pin 952 inserted position could be transferred from a preoperative planning system (such as through use of a bone model or predetermined apparatus 100 settings) to the apparatus is through use of a “setting stand” device for transferring predetermined spatial positioning information to the apparatus. A suitable device is disclosed in co-pending U.S. Patent Application Number [to be determined, attorney docket number CCF-020176] claiming priority to U.S. Provisional Patent Application No. 61/534,142, filed 13 Sep. 2011 and titled “Apparatus and Method for Transferring Predetermined Spatial Positioning Information to an Adjustable Tool”, incorporated herein by reference in its entirety.

Regardless of the manner in which the predetermined guiding relationship is provided, the user can preset the positions, lengths, orientations, and other physical characteristics of the apparatus 100 to assist in setting the elongate pin 952 into a desired landmarking relationship with the surface. In order to do so, the coupling mechanisms 114 can be manipulated to position the subordinate leg(s) 120 as desired in at least two degrees of freedom relative to the primary leg 104. When an adjustable stabilizing limb 112 is provided, it, too, may be adjusted as desired.

Once the desired adjustments have been made to the apparatus 100, the distal primary and subordinate leg ends 108 and 224 are concurrently brought into contact with the acetabular 946 surface to support the apparatus in the predetermined guiding relationship with the bone surface. This arrangement is shown in FIG. 9. As can be seen in this Figure, the guiding passageway 430 (shown partially in dotted line) extends through a substantial portion of the primary leg 104. Since the guiding passageway 430 helps to impart the insertion trajectory 102 to the elongate pin 952, the angle of a substantial portion of the primary leg 104 relative to the surface directly corresponds to the insertion trajectory in the depicted apparatus 100 and others similarly configured.

It is contemplated that the distal primary leg end 108 may contact an area of the surface directly adjacent the insertion location 956, as shown in FIG. 9, when the apparatus 100 is being supported in the predetermined guiding relationship with the surface. Regardless of the adjacency between the distal primary leg end 108 and the insertion location 956, it is also contemplated that the distal primary leg end may contact an area of the surface that is closer to the insertion location than the areas of the surface contacted by any subordinate leg 120 when the apparatus 100 is being supported in the predetermined guiding relationship with the surface. For example, as shown in FIG. 9, the distal primary leg end 108 could contact the fovia, which is directly adjacent the insertion location 956, while the distal subordinate leg ends 224 contact the acetabular rim 950, which is spaced apart from the insertion location.

Optionally, a coupling mechanism 114 having an associated subordinate leg 120 in the form of a locating fin 960 could be provided to the apparatus 100. When present, the locating fin 960 may be adjusted to contact a particular native tissue feature 962 (optionally in a mating relationship) to help orient the apparatus 100 as desired. For example, a native acetabulum 946 may have a distinctive anatomical feature (natively present or artificially provided) which can serve as a native tissue feature 962 landmark for the locating fin 960. It is anticipated that the native tissue feature 962 contacted by the locating fin 960 will be located on a portion of the acetabulum 946 which is spaced apart from the acetabular rim 950 or other locations contacted by the other subordinate legs 120—this spacing may help the locating fin and subordinate legs positively located and help steady the primary leg 104 with respect to the dictated trajectory and location. The locating fin 960 could instead be substantially rigidly attached to the primary leg 104 without an associated coupling mechanism 114.

Optionally, the primary leg 104 may guide longitudinal translational movement (e.g., through use of a guiding passageway 430) of a pilot drill (not shown) into the surface at the dictated trajectory and location when the apparatus 100 is being supported in the predetermined guiding relationship with the surface. In this manner, the pilot drill can drill a pilot hole at the dictated trajectory and insertion location in the surface to prepare the surface for insertion of the elongate pin 952.

Whether or not a pilot hole is provided, the primary leg 104 may guide longitudinal translational movement of the elongate pin 952 to insertion into the surface at the dictated insertion trajectory 102 and insertion location 956 when the apparatus 100 is being supported in the predetermined guiding relationship with the surface. In other words, the elongate pin 952 is moved in the insertion direction 958, which directly corresponds to the insertion trajectory 102, along the guiding passageway 430 (when present) or another structure of the primary leg 104 until a distal pin end 964 contacts the surface to be penetrated (the surface of the acetabulum 946 in FIG. 9). The distal pin end 964 is then inserted into the surface along the insertion trajectory 102 until the elongate pin 952 achieves a desired landmarking relationship with the surface. The apparatus 100 can then be removed from the surgical field and the surgical procedure can continue.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the described apparatus 100 are merely illustrative; one of ordinary skill in the art could readily determine any number of devices, sequences of steps, or other means/options for dictating the insertion trajectory and/or insertion location as described. Any of the described structures and components could be integrally formed as a single piece or made up of separate sub-components, with either of these formations involving any suitable stock or bespoke components and/or any suitable material or combinations of materials. It is contemplated that the apparatus 100 may be reusable (optionally sterilizable). Although an elongate pin 952 is used as an example of a structure to be inserted into a surface through guidance of the apparatus 100, any other suitable structures could be guided into position upon, or engagement with, the surface, such as, but not limited to, a bovie cauterizer, an electrode, a drill (e.g., for drilling fastener holes), a marking pen, or any other suitable structure. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. The mating relationships formed between the described structures need not keep the entirety of each of the “mating” surfaces in direct contact with each other but could include spacers or holdaways for partial direct contact, a liner or other intermediate member for indirect contact, or could even be approximated with intervening space remaining therebetween and no contact. Various structures are described as being movably attached to the primary leg 104 via coupling mechanisms 114 allowing optional movement in at least two degrees of freedom, but these structures could instead be fixedly attached to the primary leg or movably attached via any suitable attachment means, and the structures need not all be attached to the primary leg using the same attachment means. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. An apparatus for dictating an insertion trajectory and an insertion location for insertion of an elongate pin into a surface, the apparatus comprising:
a primary leg having longitudinally spaced proximal and distal primary leg ends, the distal primary leg end being configured for direct contact with the surface;
at least one subordinate leg having longitudinally spaced proximal and distal subordinate leg ends, the proximal subordinate leg end being movably attached to the primary leg by a coupling mechanism allowing at least two degrees of freedom of motion of the subordinate leg relative to the primary leg; wherein
the distal subordinate leg end is configured for contact with the surface concurrently with contact of the distal primary leg end with the surface, to support the apparatus in a predetermined guiding relationship with the surface during use of the apparatus;
the subordinate leg is adjusted relative to the primary leg to impart the dictated insertion trajectory and insertion location to the apparatus; and
the primary leg guides longitudinal translational movement of the elongate pin to insertion into the surface at the dictated insertion trajectory and insertion location when the apparatus is being supported in the predetermined guiding relationship with the surface.

2. The apparatus of claim 1, wherein first and second subordinate legs are provided, each having longitudinally spaced proximal and distal subordinate leg ends, the proximal subordinate leg end of each of the first and second subordinate legs being movably attached to the primary leg by a coupling mechanism allowing at least two degrees of freedom of motion of the respective first or second subordinate leg relative to the primary leg; wherein
each distal subordinate leg end is configured for contact with the surface concurrently with contact of the distal primary leg end with the surface, to create a tripod support of the apparatus in a predetermined guiding relationship with the surface during use of the apparatus;
the first and second subordinate legs are adjusted relative to the primary leg to impart the dictated insertion trajectory and insertion location to the apparatus; and
the primary leg guides longitudinal translational movement of the elongate pin to insertion into the surface at the dictated insertion trajectory and insertion location when the apparatus is being supported in the predetermined guiding relationship with the surface.

3. The apparatus of claim 1, wherein the primary leg is substantially larger in at least two dimensions than corresponding dimensions of the subordinate leg.

4. The apparatus of claim 1, wherein the primary leg includes a guiding passageway extending longitudinally at least partially therethrough, and the elongate pin passes through at least a portion of the guiding passageway to attain the dictated insertion trajectory and insertion location with respect to the surface.

5. The apparatus of claim 4, wherein the primary leg includes a plurality of guiding passageways extending longitudinally at least partially therethrough, and at least a portion of the elongate pin passes through at least a portion of a chosen one of the plurality of guiding passageways to attain the dictated insertion trajectory and insertion location with respect to the surface, the choice of the chosen one of the plurality of guiding passageways bearing a direct positional relationship with the dictated insertion location.

6. The apparatus of claim 1, wherein the primary leg includes a stabilizing limb branching outward at an angle from a principal body of the primary leg, the stabilizing limb includes the distal primary leg end at a location spaced apart from the principal body of the primary leg, and the elongate pin is guided to insertion into the surface at the dictated insertion trajectory and insertion location by the principal body of the primary leg.

7. The apparatus of claim 1, wherein the coupling mechanism is configured for both longitudinal and rotational movement with respect to the primary leg.

8. The apparatus of claim 1, wherein the subordinate leg is connected to the coupling mechanism for pivoting movement of the subordinate leg with respect to the primary leg.

9. The apparatus of claim 1, wherein at least one of the distal primary leg end and the distal subordinate leg end of the subordinate leg includes a projection which penetrates into the surface to help maintain position of the respective primary or subordinate leg with respect to the surface.

10. The apparatus of claim 1, wherein the surface is a body tissue of a patient.

11. The apparatus of claim 10, being sterilizable and reusable for multiple patients.

12. The apparatus of claim 10, wherein the surface is a pelvis of a patient.

13. The apparatus of claim 12, wherein the distal primary leg end contacts a fovia of an acetabulum, and the distal subordinate leg end contacts a rim of the acetabulum to support the apparatus in the predetermined guiding relationship with the surface during use of the apparatus.

14. The apparatus of claim 1, including a depth control feature for at least one of indicating and limiting a depth to which the elongate pin is inserted into the surface.

15. The apparatus of claim 1, wherein at least one of the insertion trajectory and the insertion location is chosen to provide a desired elongate pin inserted position predetermined with reference to a multi-dimensional image of the surface.

16. The apparatus of claim 1, wherein the angle of a substantial portion of the primary leg relative to the surface directly corresponds to the insertion trajectory.

17. The apparatus of claim 1, wherein the primary leg guides longitudinal translational movement of a pilot drill into the surface at the dictated trajectory and location when the apparatus is being supported in the predetermined guiding relationship with the surface, the pilot drill being configured to drill a pilot hole at the dictated insertion trajectory and insertion location in the surface, the elongate pin being inserted into the pilot hole after removal of the pilot drill from the pilot hole.

18. The apparatus of claim 1, wherein the subordinate leg is manually adjusted relative to the primary leg to dictate at least one of the insertion trajectory and the insertion location.

19. The apparatus of claim 1, wherein the subordinate leg has a variable length, and the longitudinal distance between the proximal and distal subordinate leg ends is adjustable to help dictate at least one of the insertion trajectory and the insertion location.

20. The apparatus of claim 1, wherein the coupling mechanism includes at least one detent feature configured to facilitate discrete manual adjustment of the subordinate leg relative to the primary leg in at least one degree of freedom.

21. The apparatus of claim 1, wherein the distal primary leg end contacts an area of the surface directly adjacent the insertion location when the apparatus is being supported in the predetermined guiding relationship with the surface.

22. The apparatus of claim 1, wherein the distal primary leg end contacts an area of the surface that is closer to the insertion location than the areas of the surface contacted by any subordinate leg when the apparatus is being supported in the predetermined guiding relationship with the surface.

23. A method for inserting an elongate pin into a bone surface, the method comprising the steps of:
providing a primary leg having longitudinally spaced proximal and distal primary leg ends;
providing at least one subordinate leg, each subordinate leg having longitudinally spaced proximal and distal subordinate leg ends;
movably attaching the proximal subordinate leg end to the primary leg by a coupling mechanism allowing at least two degrees of freedom of motion of the subordinate leg relative to the primary leg;
adjusting the subordinate leg in at least two degrees of freedom relative to the primary leg to impart the dictated insertion trajectory and insertion location to the primary leg;
contacting the bone surface with the distal primary leg end;
contacting the bone surface with the distal subordinate leg end concurrently with contact of the distal primary leg end with the bone surface;
supporting the apparatus in a predetermined guiding relationship with the bone surface during use of the apparatus via contact between the distal primary and subordinate leg ends with the bone surface;
dictating at least one of an insertion location and an insertion trajectory of the elongate pin relative to the bone surface by maintaining the primary leg in a predetermined position relative to the bone surface;
guiding, with the primary leg, longitudinal translational movement of the elongate pin to insertion into the surface at the dictated insertion trajectory and insertion location when the apparatus is being supported in the predetermined guiding relationship with the surface;
contacting the bone surface with a distal end of the elongate pin at the insertion location; and
inserting the distal end of the elongate pin into the bone surface along the insertion trajectory.

24. The method of claim 23, wherein the step of providing at least one subordinate leg includes the step of providing first and second subordinate legs, each having longitudinally spaced proximal and distal subordinate leg ends; and wherein the method includes the steps of:
movably attaching the proximal subordinate leg end of each of the first and second subordinate legs to the primary leg by a coupling mechanism allowing at least two degrees of freedom of motion of the respective first or second subordinate leg relative to the primary leg;
adjusting the first and second subordinate legs in at least two degrees of freedom relative to the primary leg to impart the dictated insertion trajectory and insertion location to the primary leg;
contacting the bone surface with each distal subordinate leg end concurrently with contact of the distal primary leg end with the bone surface; and
creating a tripod support of the apparatus in a predetermined guiding relationship with the bone surface during use of the apparatus via contact between the distal primary and subordinate leg ends with the bone surface.

25. The method of claim 23, wherein the primary leg includes a guiding passageway extending longitudinally at least partially therethrough, and the step of guiding, with the primary leg, longitudinal translational movement of the elongate pin to insertion into the bone surface at the dictated insertion trajectory and insertion location includes the step of passing at least a portion of the elongate pin through at least a portion of the guiding passageway to attain the dictated insertion trajectory and insertion location with respect to the bone surface.

26. The method of claim 23, wherein the primary leg includes a plurality of guiding passageways extending longitudinally at least partially therethrough, and the step of guiding, with the primary leg, longitudinal translational movement of the elongate pin to insertion into the bone surface at the dictated insertion trajectory and insertion location includes the steps of:
choosing a chosen one of the plurality of guiding passageways bearing a direct positional relationship with the dictated insertion location; and
passing at least a portion of the elongate pin through at least a portion of the chosen guiding passageway to attain the dictated insertion trajectory and insertion location with respect to the bone surface.

27. The method of claim 23, wherein the step of contacting the bone surface with the distal primary leg end includes the step of contacting a fovia of an acetabulum with the distal primary leg end, and wherein the step of contacting the bone surface with the distal subordinate leg end includes the step of contacting a rim of the acetabulum with the distal subordinate leg end to support the apparatus in the predetermined guiding relationship with the bone surface during use of the apparatus

28. The method of claim 23, including the step of at least one of indicating and limiting a depth to which the elongate pin is inserted into the bone surface.

29. The method of claim 23, wherein the step of adjusting the subordinate leg in at least two degrees of freedom relative to the primary leg to impart the dictated insertion trajectory and insertion location to the primary leg includes the step of adjusting the subordinate leg responsive to a multi-dimensional image of the bone surface to impart a predetermined insertion trajectory and a predetermined insertion location to the primary leg.

30. The method of claim 23, wherein the subordinate leg has an adjustable length, and including the step of changing the longitudinal distance between the proximal and distal subordinate leg ends of the chosen subordinate leg to help dictate at least one of the insertion trajectory and the insertion location.

31. The method of claim 23, wherein the coupling mechanism includes at least one detent feature, and including the step of facilitating discrete manual adjustment of the subordinate leg relative to the primary leg in at least one degree of freedom using the detent feature.

32. The method of claim 23, wherein the step of contacting the bone surface with the distal primary leg end includes the step of contacting an area of the bone surface directly adjacent the insertion location with the distal primary leg end when the apparatus is being supported in the predetermined guiding relationship with the surface.

33. The method of claim 23, wherein the step of contacting the bone surface with the distal primary leg end includes the step of contacting an area of the surface with the distal primary leg end that is closer to the insertion location than the area of the surface contacted by any subordinate leg when the apparatus is being supported in the predetermined guiding relationship with the surface.

34. The method of claim 23, including the steps of:
providing an adjustment aid tool; and
interacting at least one of the primary leg and the subordinate leg with the adjustment aid tool to facilitate dictation of at least one of the insertion trajectory and the insertion location.
